(19) United States
(12) Patent Application Publication
(10) Pub. No.: US 2007/0280915 A1
(43) Pub. Date: Dec. 6, 2007

(54) Combination Therapies Employing A Composition Comprising A Hmg Coa Reductase Inhibitor And A Vitamin B6 Related Compound

(75) Inventors: Albert Friesen, Winnipeg (CA); Ahmad Khalil, Winnipeg (CA); Marjorie Zettler, Winnipeg (CA)

Correspondence Address: MERCHANT & GOULD PC, P.O. BOX 2903, MINNEAPOLIS, MN, 55402-0903, US

(73) Assignee: Medicure International Inc., Winnipeg (CA)

(21) Appl. No.: 10/584,332

(22) PCT Filed: Dec. 23, 2004
(86) PCT No.: PCT/CA04/02196

§ 371 Date: Mar. 7, 2007

Related U.S. Application Data
(60) Provisional application No. 60/531,605, filed on Dec. 23, 2003.
(60) Provisional application No. 60/586,215, filed on Jul. 9, 2004.

(51) Int. Cl. A61K 31/675; A61K 31/366; A61K 31/404; A61K 31/505

(52) U.S. Cl. 424/94.1; 514/275; 514/419; 514/460

(57) ABSTRACT

The present invention provides pharmaceutical compositions comprising a HMG CoA reductase inhibitor and a vitamin B6 related compound and methods for using the pharmaceutical compositions for reducing the risk of cardiovascular and other diseases.

### FIELD OF INVENTION

This invention generally relates to combination therapies employing HMG Co A reductase inhibitors and uses thereof.

### BACKGROUND

According to the American Heart Association in 2000, 39.4% of deaths were from cardiovascular disease. The risk of developing heart disease and indirectly stroke, increases steadily as blood cholesterol values rise. Elevated blood cholesterol levels are also associated with an increased risk of developing diabetes. The desirable blood levels are <200 mg/dL. Borderline acceptable levels are in the range of 200-239 mg/dL and high risk begins at 240 md/dL or greater. It is estimated that some 102.3 million Americans have high cholesterol numbers.

Hypercholesterolemia is known to affect the responsiveness of various blood vessels to endogenous and exogenous vasoactive agents. Of particular interest is the increased responsiveness to vasoconstrictors, e.g. 5-hydroxy tryptamine and noradrenaline, and the decreased reactivity towards vasodilators, e.g. acetylcholine and nitric oxide. This together with the development of arteriosclerosis plays an important role in the progression of many cardiovascular-related disorders, such as hypertension, stroke, restinosis, late vein graft failure and coronary artery disease.

Presently hypercholesterolemia is treated primarily with lipid lowering drugs such as statins, bile salt sequestrants, fibrates or niacin. While statins are arguably the most effective lipid lowering drugs available, the use of statins in combination with other drugs, such as protease inhibitors (e.g. norvir), acetaminophen, cyclosporine, mibefradil, azole fungicides, macrolide antibiotics, and warfarin, is limited due to adverse drug-drug reactions, including most significantly, the inhibition of hepatic cytochrome P450 enzymes, which are responsible for the metabolism of drugs in the liver.

In contrast, vitamin B6 which also has lipid lowering properties, is a well tolerated drug with no significant side effects (Brattstrom et al, Pyroxidine reduces cholesterol and low-density lipoprotein and increases antithrombin III activity in 80 year old men with low plasma pyridoxal 5-phosphate, Scand J Clin Lab Invest, 1990, 50:873). Several vitamin B6 derivatives also have lipid-lowering properties. For example, U.S. Pat. No. 6,066,659 teaches the use of vitamin B6 (pyridoxine), pyridoxal and pyridoxamine derivatives for the treatment of hyperlipidemia and atherosclerosis. German Patent DE 24 61 742 C2 teaches the use of pyridoxal, pyridoxol, and pyridoxamine-5′phosphoric acid esters for treating hyperlipidemia. Supplementation with magnesium pyridoxal-5′-phosphate glutamate, has also been shown to reduce lipid levels (Khayyal et al, Effect of magnesium pyridoxal 5-phosphate glutamate on vascular reactivity in experimental hypercholesterolemia, Drugs Exp Clin Res. 1998, 24:29-40).

In addition to lipid lowering properties, vitamin B6 and its metabolites, such as pyridoxal-5′-phosphate, are useful in the treatment of cardiovascular or related disease, for example, myocardial ischemia and ischemia reperfusion injury, myocardial infarction, cardiac hypertrophy, hypertension, congestive heart failure, heart failure subsequent to myocardial infarction, vascular disease including atherosclerosis, and diseases that arise from thrombotic and prothrombotic states in which the coagulation cascade is activated.

Previous disclosures have taught the optional use of vitamin B6 (pyroxidine) with a cholesterol-lowering agent wherein the inclusion of vitamin B6 was directed to decreasing homocysteine levels. For example, U.S. Pat. No. 6,576,256 discloses a method of treating a patient with elevated cardiovascular risk by the use of a HMG CoA reductase inhibitor with an inhibitor for the renin-angiotension system, aspirin and optionally vitamin B6 (pyridoxine). US Patent Application No. 20030049314 discloses a formulation for treating a patient with elevated cardiovascular risk comprising a combination of an HMG Co A reductase inhibitor, an ACE inhibitor, aspirin and optionally vitamin B6. US Patent Application No. 20030068399 discloses an orally administrable pharmaceutical dosage form for treating a patient at elevated cardiovascular risk comprising a combination of comprising a combination of an HMG Co A reductase inhibitor, an inhibitor for the renin-angiotension system, aspirin and optionally vitamin B6. However, there are currently no combination therapies which employ a vitamin B6 related compound as a lipid-lowering agent in combination with a HMG Co A reductase inhibitor.

The use of statins in combination with other drugs, and consequently the potential for additive therapeutic benefits, has been limited because of hepatotoxicity. There are currently no combination therapies for treating and preventing hypercholesterolemia and related disorders such as cardiovascular disease and diabetes which do not induce adverse drug reactions and which are suitable for persons susceptible to drug-induced heptotoxicity. Accordingly, there is a need for new pharmaceutical compositions and methods of treatment which overcome the limitations of the current therapies involving statins.

### SUMMARY OF INVENTION

The present invention provides a pharmaceutical composition comprising: (a) a HMG CoA reductase inhibitor; (b) a vitamin B6 related compound; and (c) a pharmaceutically acceptable carrier.

In one embodiment of the invention, the HMG CoA reductase inhibitor is selected from a group consisting: pravastatin, lovastatin, fluvastatin, atorvastatin, simvastatin, rosuvastatin, velostatin, fluindostatin, and a mixture thereof.

In another embodiment of the invention, the vitamin B6 related compound is selected from a group consisting: pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

The present invention also provides a method for treating a patient at risk of cardiovascular disease comprising administering a therapeutically effective dose of the pharmaceutical composition comprising: (a) a HMG CoA reductase inhibitor; (b) a vitamin B6 related compound; and (c) a pharmaceutically acceptable carrier.

In an embodiment, the method is for treating a patient at risk of cardiovascular disease. In another embodiment, the method is for treating the patient susceptible to hepatotoxicity.

The cardiovascular disease may be selected from a group consisting: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), deep vein thrombosis (DVT), Kawazaki disease, restinosis, late vein graft failure and heart transplant.

The present invention also provides a method for treating a patient at risk of diabetes comprising administering a therapeutically effective dose of the pharmaceutical composition comprising: (a) a HMG CoA reductase inhibitor; (b) a vitamin B6 related compound; and (c) a pharmaceutically acceptable carrier.

The present invention also provides a method for treating a patient at risk of Alzheimer's disease comprising administering a therapeutically effective dose of the pharmaceutical composition comprising: (a) a HMG CoA reductase inhibitor; (b) a vitamin B6 related compound; and (c) a pharmaceutically acceptable carrier.

The present invention also provides a method for treating a patient at risk of osteoporosis comprising administering a therapeutically effective dose of the pharmaceutical composition comprising: (a) a HMG CoA reductase inhibitor; (b) a vitamin B6 related compound; and (c) a pharmaceutically acceptable carrier.

The dose of the HMG CoA reductase inhibitor may be between 0.1 and 1000 mg per day. The dose may be 10 mg per day.

The dose of the vitamin B6 related compound may be between 0.1 to 50 mg/kg per day. The dose of vitamin B6 related compound may be between 1 to 15 mg/kg per day.

The present invention further provides a method of treating or preventing hypercholesterolemia in a patient comprising administering a therapeutically effective dose of a vitamin B6 related compound wherein the vitamin B6 related compound is selected from a group consisting: pyridoxal-5′-phosphate, a 3-acylated analogue of pyridoxal, a 3-acylated analogue of pyridoxal-4,5-aminal, a pyridoxine phosphate analogue, and a mixture thereof.

### BRIEF DESCRIPTION OF THE FIGURES

FIG. 1 comprises line graphs 1(a) to 1(f), illustrating the decrease in the fluorescence of the metabolic products (CHC, 7-HC, HFC, fluorescein, AHMC and quinolinol) measured in the CYP inhibition assays as a function of pyridoxal 5′-phosphate concentration.

FIG. 2 comprises line graphs 2(a) and 2(b) illustrating the inhibition of the catalytic activity of CYP1A2 (metabolism of CEC to CHC) as a function of Furafylline and P5P concentration respectively.

FIG. 3 comprises line graphs 3(a) and 3(b) illustrating the inhibition of the catalytic activity of CYP2A6 (metabolism of coumarin to 7-HC) as a function of Tranylcypromine and P5P concentration respectively.

FIG. 4 comprises line graphs 4(a) and 4(b) illustrating the inhibition of the catalytic activity of CYP2B6 (metabolism of EFC to HFC) as a function of Tranylcypromine and P5P concentration respectively.

FIG. 5 comprises line graphs 5(a) and 5(b) illustrating the inhibition of the catalytic activity of CYP2C8 (metabolism of DBF to Fluorescein) as a function of Quercetin and P5P concentration respectively.

FIG. 6 comprises line graphs 6(a) and 6(b) illustrating the inhibition of the catalytic activity of CYP2C9 (metabolism of MFC to HFC) as a function of Sulfaphenazole and P5P concentration respectively.

FIG. 7 comprise line graphs illustrating 7(a) and 7(b) the inhibition of catalytic activity of CYP2C19 (metabolism of CEC to CHC) as a function of Tranylcypromine and P5P concentration respectively.

FIG. 8 comprises line graphs 8(a) and 8(b) illustrating the inhibition of the catalytic activity of CYP2D6 (metabolism of AMMC to AHMC) as a function of Quinidine and P5P concentration respectively.

FIG. 9 comprises line graphs 9(a) and 9(b) illustrating the inhibition of the catalytic activity of CYP2D6 (metabolism of MFC to HFC) as a function of Diethyldithiocarbamic acid (DDTC) and P5P concentration respectively.

FIG. 10 comprises line graphs 10(a) and 10(b) illustrating the inhibition of the catalytic activity of CYP3A4 (metabolism of BFC to HFC) as a function of Ketoconazole and P5P concentration respectively.

FIG. 11 comprises line graphs 11(a) and 11(b) illustrating the inhibition of the catalytic activity of CYP3A4 (metabolism of BQ to Quinolinol) as a function of Ketoconazole and P5P concentration.

FIG. 12 comprises line graphs illustrating the area under the curve CK-MB values fitted to a log-normal distribution for patients treated with P5P (A) and placebo (B).

### DETAILED DESCRIPTION OF THE INVENTION

The causal association between elevated low density lipoproteins (LDL) levels and the risk for developing cardiovascular disease is well established. Reducing elevated LDL levels have been shown to reduce the incidence of cardiovascular events, including transient ischemic attacks and indirectly strokes, and to reduce mortality. More recently, elevated LDL levels have been correlated with an increased risk for developing diabetes. The inventors of the present invention have discovered that statins and certain vitamin B6 related compounds in combination reduce the risk of cardiovascular disease and diabetes in a synergistic manner with substantially no incidence of hepatotoxicity.

Statins are highly effective lipid lowering agents. However, statin use is associated with numerous problems including drug-drug interactions, and hepatoxicity. The inventors have discovered that vitamin B6 related compounds such as P5P are effective for both improving lipid levels in its own right and are effective in ameliorating some of the problems associated with statin therapy.

The inventors have discovered that combining statins with a vitamin B6 related compound provides synergistic lipid reduction with no adverse drug-drug interactions. Statins taken in combination with other drugs will cause a drug-drug interaction that will inhibit hepatic CYP-450. This class of enzymes is primarily responsible for the metabolism of drugs in the liver. P5P and related compounds are co-enzymes for many enzymes and do not inhibit these liver enzymes and therefore, does not exacerbate the negative effects associated with the metabolism of statin.

The inventors have further discovered that co-administration of vitamin B6 related compounds helps to mitigate statin induced hepatoxicity. For example, an increase in the alanine transferase marker has been observed during statin therapy. This indicates potential hepatotoxicity. P5P and related compounds do not increase alanine transferase levels in the liver and therefore are not themselves hepatotoxic. Taken in combination with a statin, P5P and related compounds provide beneficial therapy without exacerbating the incidence and severity of hepatoxicity generally associated with statin treatment.

In addition to hepatotoxicity, co-administration of P5P and related compounds tend to improve surgical outcomes and reduce the incidence and severity of myocardial injury following PCI.

Furthermore, the pharmaceutical compositions according to the present invention reduce the risk of cardiovascular disease and diabetes. The pharmaceutical compositions according to the present invention can also be used to reduce the risk of Alzheimer's disease and osteoporosis.

With respect to alkaline phosphatase, P5P and related compounds are natural substrates for this compound. Alkaline phosphatase is implicated in bone mineralization. The link between P5P and alkaline phosphatase has been particularly document in the study of hypophosphatasia. Low serum levels of alkaline phosphatase and a range of skeletal deformities characterize hypophosphatasia. Increasing levels of P5P will improve this disorder. In contrast, studies have shown that plasma levels of bone turnover markers including alkaline phosphatase were lower in statin treated subjects than in control subjects. Thus, in combination, statins and vitamin B6 related compounds beneficially regulate bone turnover.

With respect to secretory phospholipase A2 (PLA₂), in addition to lipid lowering properties, statins have been shown to reduce PLA₂ levels (Wiklund et al., Effects of Simvastatin and atorvastatin on inflammation marker in plasma, J. Intern Med., 2002, 251:338-347). PLA₂ has been indicated as is a strong independent risk factor for coronary heart disease (Camejo et al, Phospholipase A₂ in Vascular Disease, Circ Res. 2001, 89:298:304 at 298) and is also considered an inflammatory biomarker. PLA₂ catalyses the hydrolysis of the sn-2 ester bond in glyceroacyl phospholipids present in lipoproteins and cell membranes forming non-esterified fatty acids and lysophopholipids.

PLA₂ plays a role in several processes which increase the risk for cardiovascular disease. PLA₂ can modify circulating lipoproteins and induce the formation of LDL particles associated with increased risk for cardiovascular disease (Camejo et al., 2001, at p. 298). In arterial walls, PLA₂ can induce aggregation and fusion of matrix-bound lipoproteins and further increase their binding strength to matrix proteoglycans. PLA₂ catalyzes the release of arachidonic acid from cell membranes which is converted by cycloxygenases to thromboxanes which promote vasoconstriction and platelet adhesion. Arachidonic acid is also converted by cycloxygenases to prostaglandins which mediate inflammation, a further cardiovascular disease risk factor. Prostaglandins and other inflammatory mediators influence multiple processes, including cholesterol homeostasis and coagulation.

P5P, a vitamin B6 metabolite has been implicated in the inhibition of arachidonic acid release via PLA₂ activation (Krinshnamurthi and Kakkar, Effect of pyridoxal 5′phosphate (PALP) on human platelet aggregation, dense granule release and thromboxane B2 generation—role of Schiff base formation, Thromb Haemost. 1982, 48:136). Thus, in combination, statins and vitamin B6 related compounds beneficially regulate PLA₂ levels.

The present inventors are the first to employ a vitamin B6 related compound as an active agent for the reduction of cholesterol and PLA₂ in combination with a statin. The present inventors have discovered that the lipid lowering and PLA₂ inhibition properties of vitamin B6 related compounds are significantly greater than those for vitamin B6 and other previously disclosed vitamin B6 derivatives (see U.S. Pat. No. 6,066,659 and German patent DE 24 61 742 C2). P5P is forty times more potent in vivo as compared to pyroxidine. The inventors have also discovered that cardiovascular protective effects of statin and vitamin B6 related compounds are synergized when they are administered in combination. The inventors have further discovered that statins and vitamin B6 related compounds do not react adversely when co-administered. Vitamin B6 related compounds do not inhibit hepatic CYP enzymes and do not increase hepatic transaminases.

In light of these discoveries, the present invention provides pharmaceutical compositions and uses thereof for reducing the risk of cardiovascular disease and diabetes. The pharmaceutical compositions of the present invention are more effective than currently available combination therapies in reducing risk of cardiovascular disease. The pharmaceutical compositions ameliorate multiple risk factors including lipoproteins, homocysteine, vasoconstriction, platelet aggregation and inflammation. Furthermore, the pharmaceutical compositions do not induce hepatotoxicity. The pharmaceutical compositions of the present invention are comprised of a HMG CoA reductase inhibitor, a vitamin B6 related compound or a pharmaceutically acceptable salt thereof, and a pharmaceutically acceptable carrier.

Examples of HMG CoA reductase inhibitors that may be used include but are not limited to pravastatin (Pravachol™), lovastatin (Mevacor™), fluvastatin (Lescol™), atorvastatin (Lipitor™), simvastatin (Zocor™), rosuvastatin (Crestor™), velostatin, and fluindostatin. Preferably, the HMG CoA reductase inhibitor is simvastatin. The term “HMG CoA reductase inhibitor” is intended to include all pharmaceutically acceptable salt, ester, and lactone forms of compounds that have HMG CoA reductase inhibitory activity.

Examples of the vitamin B6 related compound which may be used include but are not limited to pyridoxal-5-phosphate (P5P), pyridoxal, and pyridoxamine. Other vitamin B6 related compounds, which can also be used, include the 3-acylated analogues of pyridoxal, 3′acylated analogues of pyridoxal-4,5-aminal, and pyridoxine phosphonate analogues as disclosed in U.S. Pat. No. 6,585,414 and U.S. Patent Application No. 20030114424, both of which are incorporated herein by reference. Preferably, the vitamin B6 related compound will be P5P.

The 3-acylated analogue of pyridoxal includes:

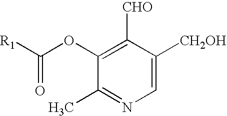

wherein,

R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or

R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or

R₁ is aryl, aryloxy, arylthio, or aralkyl, in which aryl can be substituted by alkyl, alkoxy, amino, hydroxy, halo, nitro, or alkanoyloxy.

The 3-acylated analogue of pyridoxal-4,5-aminal includes:

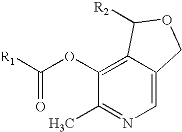

wherein,

R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or

R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or

R₁ is aryl, aryloxy, arylthio, or aralkyl, in which aryl can be substituted by alkyl, alkoxy, amino, hydroxy, halo, nitro, or alkanoyloxy;

R₂ is a secondary amino group.

The pyridoxine phosphate analogue includes:

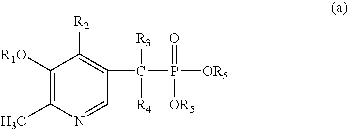

wherein,

R₁ is hydrogen or alkyl;

R₂ is —CHO—, —CH₂OH, —CH₃, —CO₂R₆ in which R6 is hydrogen, alkyl, aryl; or

R₂ is —CH₂—O alkyl in which alkyl is covalently bonded to the oxygen at the 3-position instead of R₁;

R₃ is hydrogen and R₄ is hydroxy, halo, alkoxy, alkanoyloxy, alkylamino, or arylamino; or

R₃ and R₄ are halo; and

R₅ is hydrogen, alkyl, aryl, aralkyl, or —CO₂R₇ in which R₇ is hydrogen, alkyl, aryl, or aralkyl;

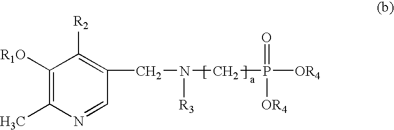

wherein,

R₁ is hydrogen or alkyl;

R₂ is —CHO, —CH₂OH, —CH₃, —CO₂R₅ in which R₅ is hydrogen, alkyl, aryl; or

R₂ is —CH₂—O alkyl in which alkyl is covalently bonded to the oxygen at the 3-position instead of R₁;

R₃ is hydrogen, alkyl, aryl, aralkyl,

R₄ is hydrogen, alkyl, aryl, aralkyl, or —CO₂R₆ in which R6 is hydrogen, alkyl, aryl or aralkyl;

n is 1 to 6; and

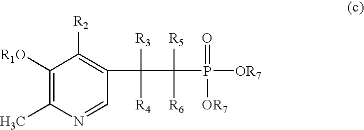

wherein,

R₁ is hydrogen or alkyl;

R₂ is —CHO—, CH₂OH—, —CH₃, —CO₂R₈ in which R₈ is hydrogen, alkyl, aryl; or

R₂ is —CH₂—O alkyl- in which alkyl is covalently bonded to the oxygen at the 3-position instead of R₁;

R₃ is hydrogen and R₄ is hydroxy, halo, alkoxy, or alkanoyloxy; or

R₃ and R₄ can be taken together to form ═O;

R₅ and R6 are hydrogen; or

R₅ and R6 are halo;

R₇ is hydrogen, alkyl, aryl, aralkyl, or —CO₂R₈ in which R₈ is hydrogen, alkyl, aryl, or aralkyl.

It is to be understood that this invention is not limited to specific dosage forms, carriers, or the like, as such may vary. It is also to be understood that the terminology used herein is for the purpose of describing particular embodiments only, and is not intended to be limiting.

Some of the compounds described herein contain one or more asymmetric centres and this may give raise to enantiomers, disasteriomers, and other stereroisomeric forms which may be defined in terms of absolute stereochemistry as (R)— or (S)—. The present invention is meant to include all such possible diasteriomers and enantiomers as well as their racemic and optically pure forms. Optically active (R)— and (S)— isomers may be prepared using chiral synthons or chiral reagents, or resolved using conventional techniques. When the compounds described herein contain olefinic double bonds or other centres of geometric symmetry, and unless specified otherwise, it is intended that the compounds include both E and A geometric isomers. Likewise all tautomeric forms are intended to be included.

As used in this specification and the appended claims, the singular forms “a,” “an”, and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

By “pharmaceutically acceptable,” such as in the recitation of a “pharmaceutically acceptable carrier,” or a “pharmaceutically acceptable salt,” is meant herein a material that is not biologically or otherwise undesirable, i.e., the material may be incorporated into a pharmaceutical composition administered to a patient without causing any undesirable biological effects or interacting in a deleterious manner with any of the other components of the composition in which it is contained.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

By an “effective” amount or a “therapeutically effective amount” of a drug or pharmacologically active agent is meant a nontoxic but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “reduce the risk of cardiovascular disease” and “reducing the risk of cardiovascular disease” as used herein refer to the reduction or elimination of an underlying cause or biomarker associated with the increased incidence of a cardiovascular event.

As used herein, “cardiovascular disease” means any disease of the heart of blood vessels. Examples of cardiovascular disease include: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), high cholesterol, deep vein thrombosis (DVT), Kawazaki disease, restinosis, late vein graft failure and heart transplant.

The terms “reduce the risk of diabetes” and “reducing the risk of diabetes” as used herein refer to the reduction or elimination of an underlying cause or biomarker associated with the increased incidence of developing insulin resistance, pre-diabetes and diabetes.

As used herein, “vitamin B6 related compound”, means any vitamin B6 precursor, metabolite, derivative, or analogue thereof but explicitly excludes: (1) vitamin B6 (pyroxidine); (2) the 5′ phosphoric acid esters of pyridoxal, pyridoxol and pyridoxamine disclosed in German Patent DE 24 61 742 C2, and (3) the pyridoxine, pyridoxal, and pyridoxamine derivatives disclosed in U.S. Pat. No. 6,066,659.

As used herein, “hepatotoxicity” includes any drug-induced liver injury. The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

Preferably, the pharmaceutical compositions of the present invention are administered orally. Preferred oral dosage forms contain a therapeutically effective unit dose of each active agent, wherein the unit dose is suitable for a once-daily oral administration. The therapeutic effective unit does of any of the active agents will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastrointestinal tract, the hepatobillary system and the renal system. Methods for determining dosage and toxicity are well known in the art with studies generally beginning in animals and then in humans if no significant animal toxicity is observed. The appropriateness of the dosage can be assessed by monitoring LDL levels, HDL levels, total cholesterol levels, triglycerides levels, and homocysteine levels. Where the dose provided does not cause LDL lipoprotein and homocysteine levels to decline to normal or tolerable levels, following at least 2 to 4 weeks of treatment, the dose can be increased.

The therapeutic effective unit dosage for the HMG CoA reductase inhibitor is between 0.1 mg and 1000 mg per day. Suitable dosage ranges for particular HMG CoA reductase inhibitors are known in the art. Typically the unit dosage will be between 5, 10, 20, 40, and 80 mg per day. Where the HMG CoA reductase inhibitor employed is simvastatin, the preferred unit dosage is 10 mg per day. The preferred unit dosage for other HMG CoA reductase inhibitors is 20 mg per day.

The preferred therapeutic effective unit dosage for the vitamin B6 related compound is between 0.1 to 50 mg/kg body weight daily. More preferably, the unit dosage will be 1 to 15 mg/kg body weight daily.

The present invention also provides a method of treating or preventing hypercholesterolemia in a patient comprising administering a therapeutically effective dose of a vitamin B6 related compound wherein the vitamin B6 related compound is pyridoxal-5′-phosphate, a 3-acylated analogue of pyridoxal, a 3-acylated analogue of pyridoxal-4,5-aminal, a pyridoxine phosphate analogue, or a mixture thereof.

Although the present invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLE ONE

### Effect of P5P on CYP Activity

The inhibitory effect of P5P on the activity of hepatic cytochrome enzymes was examined in vitro. The CYP inhibition assays used microsomes (Supersomes®, Gentest Corp., Woburn, Mass.) prepared from insect cells, each expressing an individual CYP subtype (CYP1A2, CYP2A6, CYP2B6, CYP2C8, CYP2C9, CYP2C19, CYP2D6, CYP2E1 or CYP3A4) expressed from the corresponding human CYP cDNA using a baculovirus expression vector. The microsomes also incorporated supplemental cDNA-expressed human reductase and/or cytochrome b5, as these enzymes stimulate the activity of the CYPs, allowing for a reduction in the amount of enzyme required per reaction (Gentest Corp.). The assays monitored, via fluorescence detection, the formation of a fluorescent metabolite following incubation of the microsomes with a specific CYP substrate. Two CYP substrates (7-benzyloxy-4-trifluoromethylcoumarin (BFC) and 7-benzyloxycoumarin (BQ)) were tested for CYP3A4, as this enzyme has been shown to exhibit complex inhibition kinetics. Reactions (0.2 mL) were performed in 96-well microtitre plates at 37° C. in the presence of an NADPH regenerating system [NADP+, glucose-6-phosphate (G6P), glucose-6-phosphate dehydrogenase (G6PDH)] and MgCl₂. Inhibition of metabolic product formation by pyridoxal 5′-phosphate for each enzyme was tested in the absence (0 μM) and presence of 0.0169 to 37.0 μM pyridoxal 5′-phosphate. An enzyme-selective inhibitor was also tested at 8 concentrations in each assay as a positive control. All determinations were performed in duplicate. The reagent solutions used for all of the CYP subtype assays, except CYP2C19 and CYP3A4, were prepared by MBDI. For CYP2C19 and CYP3A4, complete reagent kits purchased from Gentest Corp. (CYP2C19/CEC: Cat. No. HTS-4000, Lot No. 1; CYP3A4/BFC: Cat. No. HTS-1000, Lot No. 1) were used to perform the assays.

Assays for all enzymes were performed in the following manner: the NADPH regenerating system, appropriate buffer solution and vehicle, inhibitor (positive control) solution or test compound (pyridoxal 5′-phosphate) solution were dispensed into 96-well microtitre plates. Eight inhibitor and test compound concentrations were tested using 3-fold serial dilutions. The microtitre plates containing 0.1 mL/well of the latter mixture were pre-warmed to 37° C. in an incubator. A solution of buffer, microsomes and substrate was separately prepared and vortex mixed to disperse the protein. The reactions were initiated by the addition of the microsome/substrate solution (0.1 mL) to the wells of the microtitre plates containing the pre-warmed NADPH regenerating system, buffer and inhibitor solutions. Following specified incubation times, the reactions were stopped by the addition of 0.075 mL of a STOP solution (see below). Blank (background noise) samples were also assayed by adding the STOP solution prior to the addition of the microsome/substrate mix to the NADPH regenerating system. The amount of metabolic product formed was quantified by fluorescence detection in a fluorescence plate reader utilizing excitation and emission filters that had been optimized for the detection of each metabolite.

Prior to performing the CYP inhibition assays, the effect of pyridoxal 5′-phosphate on the fluorescence of the metabolic products measured in the assays was evaluated. The fluorescence of metabolite (one concentration, in duplicate) was measured in the absence (0 μM) and presence of 0.457 to 1000 μM pyridoxal 5′-phosphate. The concentrations and metabolic products measured were: 1 μM 3-cyano-7-hydroxycoumarin (CHC), 2.5 μM 7-hydroxycoumarin (7-HC), 2.5 μM 7-hydroxy-4-trifluoromethylcoumarin (HFC), 0.1 μM fluorescein, 10 μM 3-[2-(N,N-diethylamino)ethyl]-7-hydroxy-4-methylcoumarin (AHMC) and 10 μM quinolinol. The concentration of metabolite used was based on the expected maximum concentration of metabolite formed in the CYP inhibition assay (i.e. the concentration of metabolite measured following incubation substrate with the CYP subtype in the absence of an inhibitor). CHC is the fluorescent metabolite measured in the CYP1A2 and CYP2C19 assays. 7-HC is the fluorescent metabolite measured in the CYP2A6 assay, HFC is the fluorescent metabolite measured in the CYP2B6, CYP2C9, CYP2E1 and CYP3A4 (BFC as substrate) assays and fluorescein is the metabolite measured in the CYP2C8 assay. AHMC is the metabolite measured in the CYP2D6 assay and quinolinol is measured in the CYP3A4 (BQ as substrate) assay.

Pyridoxal 5′-Phosphate Solution—pyridoxal 5′-phosphate monohydrate (P5P, Lot No. 00001448) was supplied as powder. The concentrations of all pyridoxal 5′-phosphate solutions are based on the anhydrous molecular weight (247.15 g/mole) corrected for a potency factor of 0.9019.

For the determination of the effect of pyridoxal 5′-phosphate on metabolite fluorescence, a stock solution of pyridoxal 5′-phosphate, at a concentration of 50 mM, was freshly prepared in distilled water. Since pyridoxal 5′-phosphate is acidic in aqueous solution, the pH of the solution was adjusted to 7.0 with 1 N NaOH. The solution of pyridoxal 5′-phosphate was added to the wells of the microtitre plate starting with a 50-fold dilution to 1000 μM, followed by 3-fold serial dilutions to: 333, 111, 37.0, 12.3, 4.12, 1.37 and 0.457 μM.

For the CYP subtype inhibition assays, a stock solution of pyridoxal 5′-phosphate, at a concentration of 50 mM, was freshly prepared in distilled water (pH adjusted to 7.0 with 1 N NaOH). The solution of pyridoxal 5′-phosphate was diluted with distilled water to 111 μM and then added to the wells of the microtitre plate starting with a 3-fold dilution to 37.0 μM, followed by 3-fold serial dilutions to: 12.4, 4.12, 1.37, 0.457, 0.152, 0.0508 and 0.0169 μM.

Data Analysis—The mean of the duplicate fluorescent signals in the presence and absence (vehicle control) of each compound was calculated and corrected for the background noise. Percent inhibition was calculated as the difference in the corrected fluorescent signals in the absence and presence of the compound, divided by the corrected fluorescent signal in the absence of compound, multiplied by 100%. The concentration of the inhibitor or pyridoxal 5′-phosphate, where appropriate, which inhibited metabolite formation by 50% (IC₅₀) was calculated by nonlinear regression analysis (sigmoidal dose-response curve) of the % Inhibition versus Log concentration data using GraphPad Prism software (Version 3.00, GraphPad Software, Inc., San Diego, Calif.).

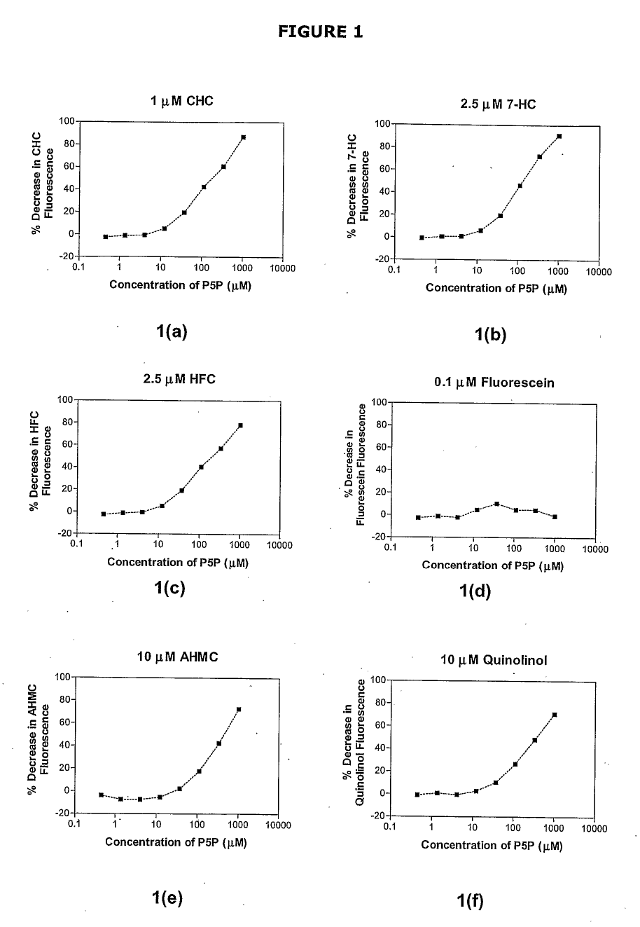

Results—The effect of pyridoxal 5′-phosphate on the fluorescence of the various metabolic products measured in the CYP inhibition assays was determined. As evident in FIG. 1, pyridoxal 5′-phosphate significantly quenched (decreased) the fluorescence of five the six metabolites measured in the assays (CHC, 7-HC, 7-HFC, AHMC and quinolinol) at concentrations of >37 μM. Pyridoxal 5′-phosphate (up to 1000 μM) did not affect the fluorescence of fluorescein, the metabolic product measured following the metabolism of dibenzylfluorescein by the CYP2C8 enzyme. The inhibitory effect of pyridoxal 5′-phosphate on CYP catalytic activity was tested over the concentration range of 0.0169 to 37 μM.

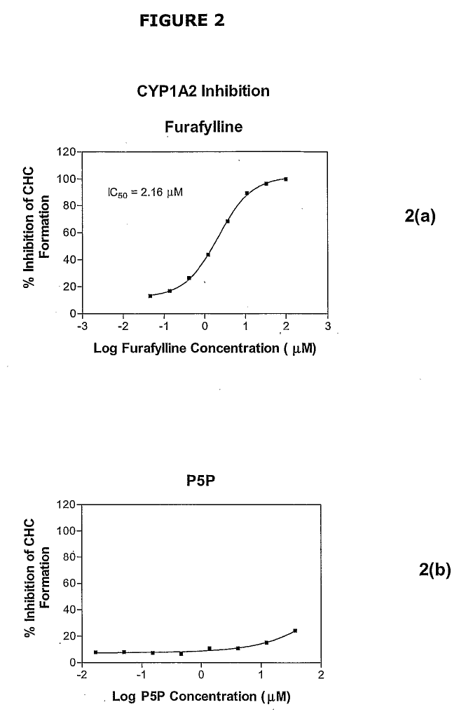

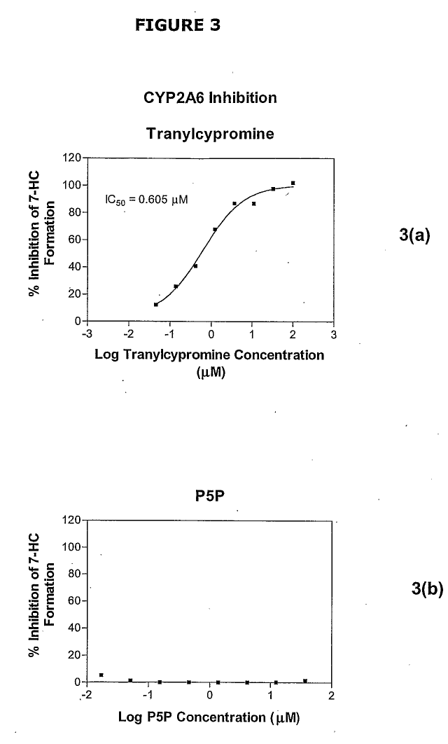

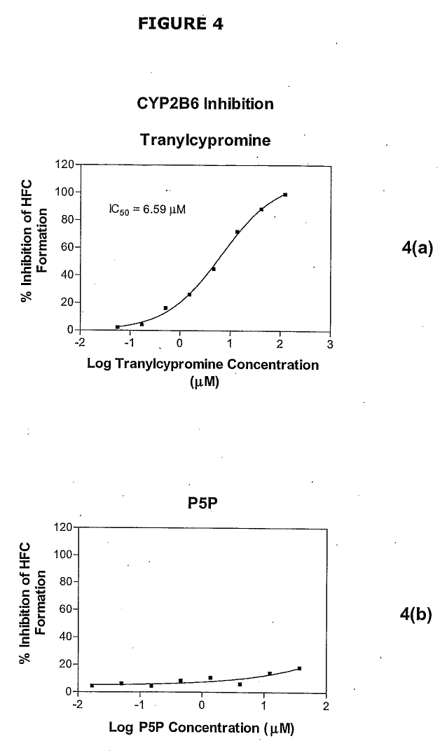

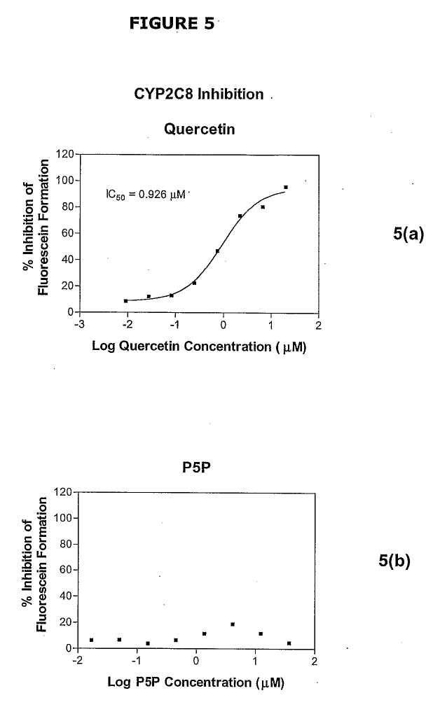

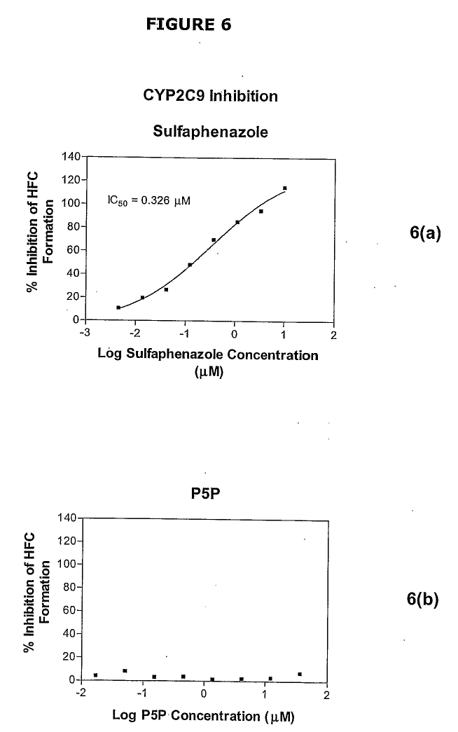

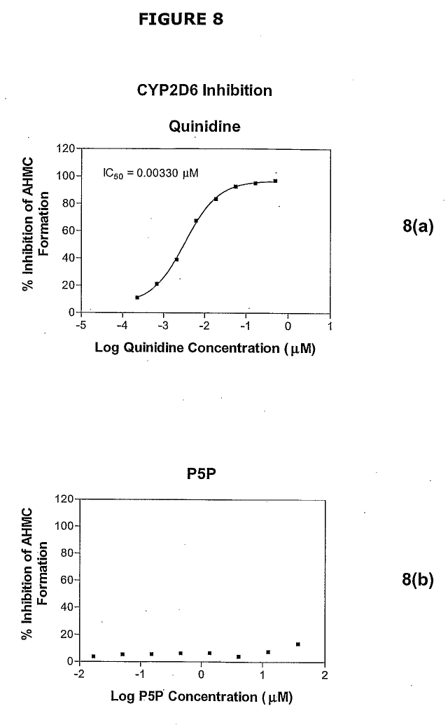

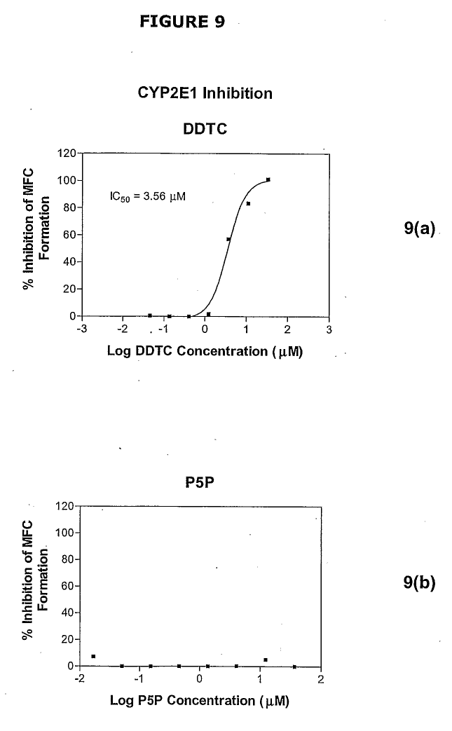

The results from the incubation of the known inhibitors and pyridoxal 5′-phosphate with each of the CYP subtypes (CYP1A2, CYP2A6, CYP2B6, CYP2C8, CYP2C9, CYP2C19, CYP2D6, CYP2E1 and CYP3A4) are depicted graphically in FIGS. 2 to 11. The observed IC₅₀ values for the various CYP inhibitors are similar to those obtained previously in our laboratory during assay validation (for CYP1A2, CYP2A6, CYP2B6, CYP2C8, CYP2C9, CYP2D6 and CYP2E1) and are similar to those determined by the supplier (for the CYP2C19 and CYP3A4 assay kits) (Table 1). These data indicate that enzyme activity was not compromised in any of the assays.

TABLE 1
Summary of IC50 values estimated for the known inhibitors of each
CYP subtype, and for pyridoxal 5′-phosphate

| | | | | | Pyridoxal |
|---|---|---|---|---|---|
| | | | Expected | Observed | 5′- |
| | | | Inhibitor | Inhibitor | phosphate |
| CYP | | Inhibitor | IC₅₀ | IC₅₀ | IC₅₀ |
| Subtype | Substrate | (positive control) | (μM) | | |
| CYP1A2 | CEC | Furafylline | 2.22 ± 1.15^{a} | 2.16 | na |
| CYP2A6 | Coumarin | Tranylcypromine | 0.990 ± 0.209^{a} | 0.605 | na |
| CYP2B6 | EFC | Tranylcypromine | 8.82 ± 2.43^{a} | 6.59 | na |
| CYP2C8 | DBF | Quercetin | 1.40 ± 0.240^{a} | 0.926 | na |
| CYP2C9 | MFC | Sulfaphenazole | 0.401 ± 0.116^{a} | 0.326 | na |
| CYP2C19 | CEC | Tranylcypromine | 0.825^{b} | 0.948 | 32.6 |
| CYP2D6 | AMMC | Quinidine | 0.00544 ± 0.00173^{a} | 0.00330 | na |
| CYP2E1 | MFC | DDTC | 5.06 ± 1.96^{a} | 3.56 | na |
| CYP3A4 | BFC | Ketoconazole | 0.018^{b} | 0.0409 | ≅37 |
| | BQ | | 0.400^{b} | 0.774 | >37 |

^{a}value expected from assay validation (mean ± S.D., n ≧ 8 experiments).
^{b}value determined by the supplier using all the components contained in the current lot kit.
na denotes hot applicable since concentration-dependent inhibition was not observed over the concentration range tested.

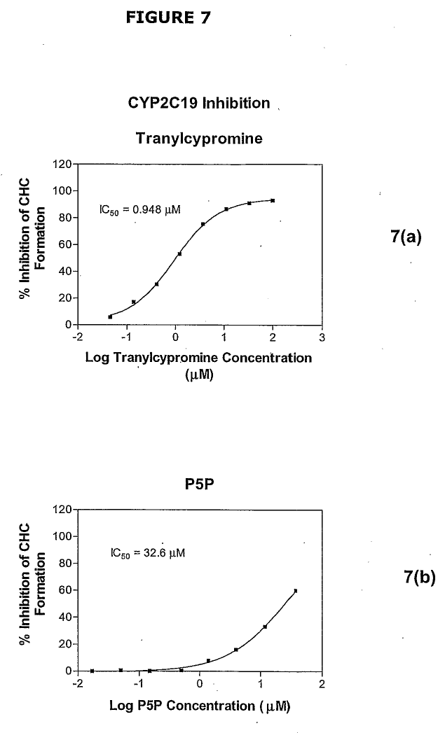

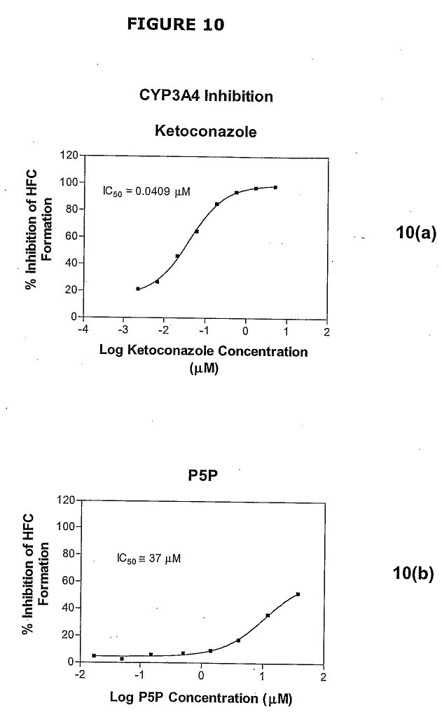

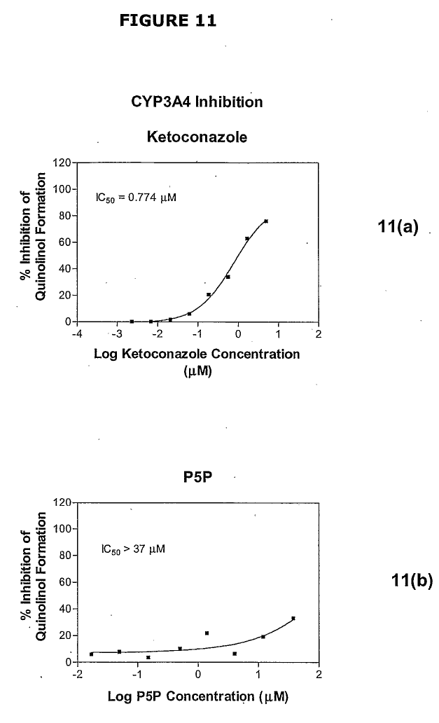

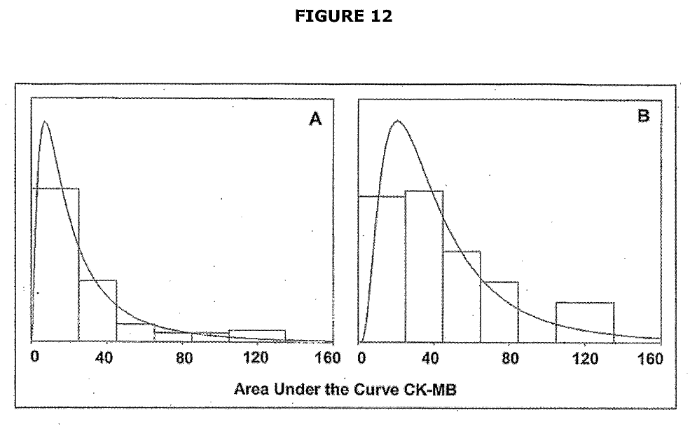

Over the concentration range tested (0.0169 to 37.0 μM), pyridoxal 5′-phosphate did not inhibit the catalytic activity of seven of the CYP enzymes: CYP1A2, CYP2A6, CYP2B6, CYP2C8, CYP2C9, CYP2D6 and CYP2E1 (FIGS. 2, 3, 4, 5, 6, 8 and 9, respectively). Pyridoxal 5′-phosphate did, however, inhibit the metabolic activity of the CYP2C19 and CYP3A4 enzyme subtypes (FIGS. 7, 10 and 11). The potency of pyridoxal 5′-phosphate was relatively similar for the CYP2C19 and CYP3A4 enzyme subtypes (IC₅₀ values of ˜33 and ˜37 μM, respectively). Pyridoxal 5′-phosphate appeared to inhibit the CYP3A4 enzyme-mediated metabolism of the substrate BFC to a slightly greater extent (IC₅₀≅37 μM) than the substrate BQ (IC₅₀>37 μM, FIGS. 10 and 11, respectively). A summary of the IC₅₀ values for pyridoxal 5′-phosphate and the known inhibitors is given in FIG. 12.

Conclusions—The compound pyridoxal 5′-phosphate did not selectively inhibit the catalytic activity of seven CYP subtypes: CYP1A2, CYP2A6, CYP2B6, CYP2C8, CYP2C9, CYP2D6 and CYP2E1, over the concentration range tested (0.0169 to 37.0 μM). Clinically relevant drug interactions would, therefore, not be expected to occur between pyridoxal 5′-phosphate and substrates of these enzymes. Pyridoxal 5′-phosphate did selectively inhibit the catalytic activity of two (CYP2C19 and CYP3A4) of the nine human CYP subtypes tested at relatively high concentrations (IC₅₀=33 μM for CYP2C19 and ≧37 μM for CYP3A4). However, based on the relatively low inhibitory potency of pyridoxal 5′-phosphate for the two CYP subtypes in vitro, the occurrence of serious drug interactions is expected to be unlikely.

### EXAMPLE TWO

### P5P and Statin Combination Therapy Reduces Myocardial Ischemic Injury Following Coronary Intervention

Methods—Study Overview: 60 patients who underwent percutaneous coronary intervention (PCI) at 4 centers were randomized in a 2:1 double-blinded fashion to treatment with P5P or placebo. Inclusion criteria required prior determination for non-urgent PCI of a single-vessel lesion(s) and identification of ≧1 of the following clinical characteristics determining high risk for procedural-related ischemic complications (Califf R M, Abdelmeguid A E, Kuntz R E, Popma J J, Davidson C J, Cohen E A, Kleiman N S, Mahaffey K W, Topol E J, Pepine C J, et al. Myonecrosis after revascularization procedures. J Am Coll Cardiol 1998; 31:241-251; The ESPRIT Investigators. Novel dosing regimen of eptifibatide in planned coronary stent implantation: a randomised, placebo-controlled trial. Lancet 2000; 356:2037-2044): presence of an acute coronary syndrome (chest pain within 48 hours of PCI), recent AMI (≦7 days), diminished epicardial blood flow, angiographic thrombus, ejection fraction ≦30%, or vein graft lesion. In addition to any general contraindication to the PCI procedure or standard concomitant therapies, major exclusion criteria were creatine kinase (CK-MB) elevation above the upper limit of normal immediately before PCI, electrocardiographic evidence of atrial fibrillation or left bundle branch block, or evidence of any clinically significant abnormal laboratory finding (transaminases, bilirubin, or alkaline phosphatase >1.5 times the upper limit of normal or serum creatinine >1.8 mg/dl). Patients with elevated troponin measurements were permitted in the study provided that the peak troponin value was reported >24 hours before scheduled PCI, with documentation of a decreasing value before revascularization. After providing informed consent, patients randomized to treatment with P5P were administered enteric-coated P5P as a 10 mg/kg oral dose >4 hours before PCI followed by 2 daily doses of 5 mg/kg orally for 14 days. Compliance and reasons for discontinued treatments were recorded for all patients.

Study end points and definitions—The primary objective of the study was to evaluate the feasibility of treatment with P5P as a cardioprotective agent in high-risk elective PCI. The primary end point of infarct size was evaluated by the trapezoidal rule (Press W H, Teukolsky S A, Vetterling W T, Flannery B P. Numerical Recipes. Cambridge, UK: Cambridge University Press, 1994:127-133.) using serial CK-MB enzyme measures performed at baseline and every 6 hours for 24 hours beginning immediately before initiation of PCI. The occurrence of myocardial ischemia within 24 hours after PCI was assessed as a secondary end point using continuous 12-lead electrocardiographic monitoring (Northeast Monitoring, Boston, Mass.). Evidence of periprocedural ischemia was defined as ST-segment depression of >100 μV within a 60-minute period of PCI, lasting ≧1 minute and separated from other episodes by 1 minute. Area under the curve ST-segment deviation was measured from the onset of the first to the last contrast injection. All cardiac markers and ST-segment monitoring data were analyzed by core laboratories blinded to treatment assignment (University of Maryland School of Medicine, Baltimore, Md.; Duke Ischemia Monitoring Laboratory, Durham, N.C.). Additional prespecified secondary end points included the 30-day composite and individual event rates of death; nonfatal infarction; new or worsening heart failure, or recurrent ischemia in addition to net clinical safety, which was defined as the absence of major adverse ischemic events; Thrombolysis In Myocardial Infarction (TIMI) major bleeding; and liver function or coagulation test abnormalities. Acute myocardial infarction (AMI) was defined as CK-MB elevation ≧3 times the upper limit of normal (upper limit of normal 7 ng/ml) and/or troponin T levels ≦1.5 times the upper limit of normal (upper limit of normal 0.1 ng/ml). If previous troponin (or CKMB) values were above the upper limit of normal, values were required to be 50% of the baseline measurement in addition to ≧2 times (≧3 times for CK-MB) the upper limit of normal to meet the definition of AMI. Routine chemistries, complete blood count, and coagulation assays were performed at baseline, 7 days, and 30 days after randomization. Peak periprocedural CK-MB and the maximum difference in troponin levels from baseline to within 24 hours after PCI were also examined.

Data collection and statistical analyses—Patients who received >1 dose of the study drug and underwent PCI were analyzed for all primary and secondary efficacy and safety end points. Patients who received >1 dose of study drug but who did not undergo PCI were excluded from the primary efficacy and ST segment monitoring analyses but were included in the safety analyses. Statistical tests were 2-sided with an a level of 0.05 and employed the intent-to-treat principle. The Wilcoxon rank-sum test was used to analyze all continuous variables. Categorical variables were compared using the Fisher's exact test with the exception of the ST-segment monitoring data, which utilized the Pearson's chi-square test. Statistical analyses were performed using SAS version 8.2 (SAS Institute, Cary, N.C.).

Results—Of the 60 patients enrolled in the study of P5P in high-risk PCI, all patients received treatment with P5P or placebo; however, 4 patients (3 P5P, 1 placebo) did not undergo planned revascularization. An additional 3 patients were excluded from the area under the curve analyses due to incomplete collection of cardiac enzyme data. As a result, 53 and 60 patients were included in the primary efficacy and 30-day clinical and/or safety analyses, respectively.

The presence of established cardiovascular disease, prior revascularization, and cardiovascular risk factors were similar between patients randomized to P5P or placebo and representative of patient populations in larger contemporary trials that studied patients with acute coronary syndromes (Table 2). Overall, the mean age of the population was 58 years, 81.7% of patients were men, and 21.7% had undergone previous PCI and/or bypass surgery. Although recent AMI as an indication for revascularization occurred more commonly among patients treated with P5P, a similar number of patients in each group presented with an acute coronary syndrome, and approximately half of all patients had elevated troponin levels before PCI.

Except for a higher incidence of reduced epicardial flow among control patients, baseline angiographic and procedural characteristics also appeared similar between treatment groups (Table 3). Administration of P5P or placebo occurred an average of 6.1 and 8.4 hours before PCI, respectively. Stent implantation was performed in 100% and 97.3% of the placebo and P5P treatment groups, respectively. Only 1 vein graft intervention was performed using distal embolic protection. Although the right coronary artery was most commonly treated in both groups, fewer patients treated with placebo underwent revascularization of a saphenous vein graft (Table 4). Procedural angiographic complications (e.g., major dissection, abrupt vessel closure) were infrequent (Table 3).

TABLE 2
Baseline clinical, electrocardiographic, and angiographic characteristics

| | P5P (n = 40) | | Placebo (n = 20) | |
|---|---|---|---|---|
| Clinical Characteristics* | | | | |
| Age (yrs) (range) | 54 | (48-66) | 59 | (55-69) |
| Men | 32 | (80) | 17 | (85) |
| Baseline troponin positive | 14/30 | (47) | 6/14 | (43) |
| Diabetes mellitus | 9 | (23) | 4 | (20) |
| Systemic hypertension | 17 | (43) | 9 | (45) |
| Hyperlipidemia (requiring | 31 | (78) | 17 | (85) |
| medical treatment or | | | | |
| LDL >130 mg/dL) | | | | |
| Current Smoker | 12 | (30) | 5 | (25) |
| Prior myocardial infarction | 14 | (35) | 9 | (45) |
| Prior PCI | 6 | (15) | 2 | (10) |
| Prior coronary bypass graft | 5 | (13) | 2 | (10) |
| surgery | | | | |
| Prior stroke or transient | 1 | (3) | 1 | (5) |
| ischemic attack | | | | |
| Peripheral vascular disease | 3 | (8) | 7 | (35) |
| Congestive heart failure | 3 | (8) | 2 | (10) |
| Qualifying electrocardiogram | | | | |
| ST-segment depression | 2 | (5) | 2 | (10) |
| ST-segment elevation | 7 | (18) | 2 | (10) |
| T-wave inversion | 6 | (15) | 4 | (20) |
| Angiographic characteristics | | | | |
| PCI performed | 37 | (93) | 19 | (95) |
| Reasons for PCI | (n = 37) | | (n = 19) | |
| Acute coronary syndrome | 9 | (24) | 5 | (25) |
| Recent AMI | 16 | (42) | 3 | (15) |
| Reduced epicardial flow | 6 | (16) | 8 | (40) |
| Thrombus | 1 | (3) | 1 | (5) |
| Congestive heart failure | 2 | (5) | 1 | (5) |
| Saphenous vein graft lesion | 4 | (11) | 2 | (10) |
| No. of coronary arteries | (n = 37) | | (n = 19) | |
| narrowed ≧50% | | | | |
| in diameter | | | | |
| 0 | 1 | (3) | 0 | |
| 1 | 19 | (48) | 14 | (70) |
| 2 | 13 | (33) | 2 | (10) |
| 3 | 5 | (13) | 3 | (15) |
| Left main | 2 | (5) | 1 | (5) |
| Left ventricular ejection | 0.50 | (0.40-0.68) | 0.56 | (0.37-0.64) |
| fraction | | | | |
| No. of coronary narrowings | (n = 37) | | (n = 19) | |
| treated | | | | |
| 1 | 26 | (70) | 15 | (79) |
| 2 | 8 | (22) | 3 | (16) |
| 3 | 3 | (8) | 1 | (5) |

Values are expressed as median (interquartile range) or number (percent)
*Patients may be double counted
LDL = low density lipoprotein

TABLE 3
Procedural and angiographic* results

| | P5P (n = 37) | | Placebo (n = 19) | |
|---|---|---|---|---|
| ≧1 stent implanted | 36 | (97) | 19 | (100) |
| Patients received GP IIb/IIIa | 29/35 | (83) | 15/19 | (79) |
| inhibitor | | | | |

Target vessel

| | | | | |
|---|---|---|---|---|
| Left anterior descending | 11 | (30) | 4 | (21) |
| Right | 14 | (38) | 11 | (58) |
| Left circumflex | 8 | (22) | 3 | (16) |
| Saphenous vein graft | 4 | (11) | 1 | (5) |

TIMI flow preprocedure

| | | | | |
|---|---|---|---|---|
| 0/1 | 3 | (8) | 4 | (22) |
| 2 | 7 | (19) | 4 | (22) |
| 3 | 27 | (73) | 10 | (56) |

TIMI flow final

| | | | | |
|---|---|---|---|---|
| 0/1 | 0 | | 0 | |
| 2 | 0 | | 1 | (5) |
| 3 | 37 | (100) | 18 | (95) |
| Diameter stenosis | 90.0 | (80.0-95.0) | 95.0 | (90.0-99.0) |
| preprocedure (%) | | | | |
| Diameter stenosis final (%) | 0 | (0-0) | 0 | (0-0) |

Procedural complications

| | | | | |
|---|---|---|---|---|
| None | 35 | (95) | 18 | (95) |
| Major dissection | 1 | (3) | 1 | (5) |
| Abrupt closure | 0 | | 0 | |
| No reflow | 0 | | 0 | |
| Thrombus formation | 0 | | 0 | |
| Side branch closure | 1 | (3) | 0 | |
| Distal embolization | 0 | | 0 | |

Values are expressed as median (interquartile range) or number (percent)
*Investigator-reported angiographic values
GP = glycoprotein

The primary end point of periprocedural infarct size measured according to median periprocedural CK-MB area under the curve was reduced from 32.9 to 18.6 ng/ml (p=0.038), reflecting a shift in the distribution of CK-MB (Table 4 and FIG. 12). Similarly, the maximum periprocedural CK-MB level was significantly lower among patients receiving P5P. By categorical classification, the occurrence of 30-day nonfatal AMI did not differ between groups (12.8% with P5P vs 10.0% with placebo, p=1.0). There were no deaths, and 30-day composite adverse event rates (death, nonfatal AMI, new and/or worsening heart failure, or recurrent ischemia) were similar (17.9% with P5P vs 15.0% with placebo, p=1.0).

Electrocardiographic ST monitoring data were available for 94.6% of the patients who underwent PCI and who received treatment (Table 4). Post-PCI ischemia occurred in approximately 15% of patients in both groups. Although lower rates of post-PCI ischemia were observed with P5P treatment (14.7% vs 17.6%, p=0.78), there were no significant differences in ischemia parameters per continuous electrocardiographic monitoring (Table 4).

TABLE 4
Perioprocedural cardiac markers and ST monitoring results

| | P5P | Placebo | p-value |
|---|---|---|---|
| Periprocedural cardiac markers | 18.6 (10.2-34.5), | 32.9 (19.4-64.3), | 0.04 |
| | 35 | 18 | |
| Area under the curve CK-MB | 1.1 (0.5-2.4), 39 | 2.0 (1.4-6.3), 19 | 0.03 |
| (ng/ml) | | | |
| Change in troponin T (ng/ml) | 0 (0 = 0.7), 36 | 0 (0-0.10), 19 | 0.65 |
| Time to peak CK-MB (h) | 11.0 (0-18.0), 36 | 14.0 (12.0-18.0), | 0.10 |
| | | 19 | |

24 h-continuous
electrocardiographic
ST-monitoring

| | | | |
|---|---|---|---|
| Duration of monitoring (h) | 22.6(20.4-23.9), | 22.4 (20.6-24.0), | — |
| | 36 | 17 | |
| Area under the curve ST deviation | 1349 (951-2,263), | 1603 (1,049-1,945), | 0.49 |
| (μV-min) | 35 | 17 | |
| Any post-PCI ischemia (%) | 14.7-34 | 17.6-17 | 0.78 |

Values are expressed as median (interquartile range) or percent followed by n (number of observations)

No safety issues related to treatment with P5P were identified. The occurrence of major bleeding (2.8% P5P vs 10.5% placebo, p=0.27) and need for blood product transfusion (2.5% P5P vs 10.00% placebo, p=0.26) was infrequent and did not significantly differ between groups. There were no apparent differences in abnormalities of routine chemistries or coagulation studies at 7 and 30 days. In both groups, however, approximately ¼ of patients discontinued drug therapy before completion of the prescribed 2 weeks (30.8% P5P vs 25.0% placebo, p=0.77). For patients taking P5P, but who did not undergo PCI (3 patients, 7.5%), the most common causes for early discontinuation were gastrointestinal intolerance followed by non-specific musculoskeletal pain.

Of the 60 patients participating in the study, 28 of the patients received adjunctive treatment with a statin in addition to P5P treatment. As shown in Table 5, the maximum periprocedural CK-MB level was reduced in patients treated with P5P and statin as compared to patients treated with placebo and statin.

TABLE 5
Effect of P5P and Statin Combination Therapy on CK-MB

| | CK-MB max (mean) | |
|---|---|---|
| Therapy | ng/ml | Sample Size |
| P5P + statin | 2.43 | 28 |
| P5P alone | 0.73 | 7 |
| Placebo + statin | 3.58 | 15 |
| Placebo alone | 2.75 | 4 |

Conclusion—In high-risk patients for periprocedural ischemic complications, statin treatment was associated with poorer outcomes following PCI. Treatment with P5P was associated with a decrease in myocardial injury, reflected by a reduction in the total amount of CK-MB released after PCI. P5P therapy was associated with a significant decrease in peak periprocedural CK-MB elevation, a significant shift in the distribution of CK-MB to lower levels (FIG. 13).

### EXAMPLE THREE

### P5P and Statin Combination Therapy Lowers Blood Pressure and Improves Lipid Levels in Hypertensive Diabetics

Overview—The study was a 14-week, open-label, forced, dose-escalation study of the efficacy and safety of P5P administered once daily over a dose range of 250 mg to 750 mg, for the treatment of mild to moderate hypertension and hyperlipidemia, in patients with coexisting diabetes mellitus.

Patient Definition—Patients meeting all of the inclusion criteria and none of the exclusion criteria were eligible for enrollment. The primary inclusion criteria were hypertension and diabetes. At screening all patients must present with or have a history of, uncomplicated, stable, mild to moderate hypertension, (supine diastolic blood pressure SUDBP ≧90 and ≦114 mm Hg) regardless of use or non-use of treatment for this disease. Additionally, at the end of the 4-week placebo lead-in period, to be eligible for single blind treatment, patients must demonstrate a mean SUDBP of ≧90 mm Hg and ≦114 mm Hg. The hypertensive patients must present with and have a history (>24 months) of diabetes mellitus (type 1 or 2), that is controlled or uncontrolled, at both screening and treatment initiation visits, to be eligible for inclusion in the study. Other than having diabetes and hypertension, patients must be in good health for their age. A history and physical examination must be within age-related normal limits, or if abnormal, considered clinically insignificant in the opinion of the investigator. Laboratory tests (CBC with differential, prothrombin and partial thrombo-plastin times, platelet count, and urinalysis and blood chemistry panel), must be normal, or if abnormal, considered clinically insignificant. Patients must have a normal ECG or, if abnormal, considered clinically insignificant.

Exclusion criteria include the presence or history (past 12 months) of accelerated or malignant hypertension, as evidenced by hemorrhage and/or exudate, and/or papilloedema on funduscopic examination. Patients having a history of angioedema of the face, lips, tongue, glottis or larynx when treated with ACE inhibitors were excluded. Patients having a diastolic BP>114 mm Hg, and/or systolic BP>200 mm Hg at screening were excluded. Patients with similar readings at the end of the placebo lead-in period are dropped from further participation in the study. During the placebo lead-in period patients who have signs of bradycardia (<45 bpm) or a resting heart rate of >100 bpm were not advanced to the next stage of the study, but rather dropped from further participation. Exclusion criteria include the presence or history (past 6 months) of MI or cerebrovascular accident and clinically significant cardiac pathology such as congestive heart failure cardio-genic shock, non-controlled arrhythmias, acute myocarditis or pericarditus, significant valvular or congestive heart disease and unstable angina pectoris; presence or evidence of atrioventricular block (second/third degree), or sick sinus syndrome, or any other conduction defect or abnormality including the presence/history of atrial fibrillation or flutter, associated with pre-excitation syndrome; clinically significant gastrointestinal disease, renal insufficiency (serum creatinine >115 umol/L), hepatic disease, or electrolyte imbalance (serum potassium <3.5 or >5.3 mmol/L, serum sodium <136->145 mmol/L); presence of bronchial asthma or chronic obstructive pulmonary disease; or the presence of any clinically significant concomitant disease that in the opinion of the investigator could interfere with the patient's participation in the study, or confound the outcome variables. Patients having any clinically significant concomitant disease that could interfere with the patient's participation in the study, or confound the outcome variables were excluded. Patients must not have a history of alcohol abuse or illicit drug use in the 12 months prior to beginning this protocol. Patients must not have a terminal illness. The patients must not have an arm circumference >41 cm. Pregnant females, women who are breast-feeding or women of child-bearing potential must use adequate contraception. Patients who have had therapy with an investigational compound within 30 days prior to entry into the study are excluded. The use of the following medications is cause for exclusion; each must be safely discontinued prior to the placebo lead-in phase of the study and must be withheld until the protocol has been completed: all diuretics and antihypertensive drugs (including beta-blockers, calcium-channel blockers, vasodilators, ACE inhibitors), MAO inhibitors, all anti-arrhythmic drugs, digitalis, major tranquilizers, antidepressants, cimetidine, corticosteroids, and antineoplastics. Patients must give informed consent. Further exclusion criteria are any of the contraindication listed in the Product Prescribing Information for Vitamin B₆ (PDR, 57, 2003:590, 926, 1894, 3221, 3237).

Treatment Schedule—Table 6 shows the study treatment schedule by clinic. Essentially, all patients complete a fixed four-week placebo lead-in, to qualify for initiation of active treatment. Eligible patients then begin treatment with 250 mg/day of P5P. This dose was administered orally on a daily basis for the next two (2) weeks, subject to dose-limiting side-effects, after which patients are force-titrated to 500 mg/day of P5P. Again, after two (2) weeks of treatment at the 500 mg/day dose level and subject to dose-limiting side-effects all patients were force-titrated to 750 mg/day of P5P and maintained at this dose level in the absence of dose-limiting side-effects for the next two (2) weeks. Thereafter, and for the final four (4) weeks of the study all patients were treated with placebo medication. Total study duration for any one patient was 14 weeks with twelve (12) clinic visits. Patients who were on anti-lipid medication prior to the initiation of the study continued their anti-lipid regime throughout the study.

TABLE 6
Study Treatment Schedule by Clinic Visit

| | Period | | | |
|---|---|---|---|---|
| | Description | Week | DAY | Visit # |
| Period | SCREENING | 0 | -21 to -3 | 1 |
| Placebo | | 1 | 0 | 2 |
| | Begin | 2 | 8-14 | 3 |
| | Lead-In | 3 | 15-21 | |
| | End | 4 | 28 | 4 |
| Active | | 4 | | |
| Treatment | Begin | 4 weeks + 3 | 31 | 5 |
| Period | | days | | |
| | 250 mg P5P | 5 | 35 | |
| | End | 6 | 42 | 6 |
| | | 6 | | |
| | Begin | 6 weeks + 3 | 45 | 7 |
| | | days | | |
| | 500 mg P5P | 7 | 49 | |
| | End | 8 | 56 | 8 |
| | | 8 | | |
| | Begin | 8 weeks + 3 | 59 | 9 |
| | | days | | |
| | 750 mg P5P | 9 | 63 | |
| | End | 10 | 70 | 10 |
| Placebo | Begin | 10 | | |
| | Lead-Out | 12 | 84 | 11 |
| | End | 14 | 98 | 12 |

Tests and Measurements—All study participants were evaluated periodically using the following tests:

Primary Efficacy Evaluations
Supine diastolic blood pressure (SUDBP) measured at specified intervals;
Triglyceride, total cholesterol, LDL cholesterol, and HDL levels measured at specified intervals.

Secondary Efficacy Evaluations
Supine systolic blood pressure (SUSBP) measured at specified intervals
Standing systolic and diastolic blood pressure (STSBP/STDBP), immediate and delayed, measured at specified intervals.

Safety Assessments
Physical examination, full and brief;
Laboratory evaluations: CBC, blood chemistries including total bilirubin, creatinine, SGOT, SGPT, alkaline phosphatase, BUN, serum glucose, total protein, electrolytes (including sodium, potassium, and chloride), and blood lipids, folic acid, and serum homocysteine levels; and urinalysis
12-lead ECG, with interpretation;
Adverse events, recorded at every visit;
Concomitant medications, recorded at every visit.

Efficacy Evaluations—In general, the following evaluations for drug efficacy are performed.
Supine systolic blood pressure (SUSBP)
Supine diastolic blood pressure (SUDBP)
Standing (IMMEDIATE) systolic blood pressure (STiSBP)
Standing (IMMEDIATE) diastolic blood pressure (STiDBP)
Standing (DELAYED, 2 minutes) systolic blood pressure (STdSBP)
Standing (DELAYED, 2 minutes) diastolic blood pressure (STdDBP)
Triglycerides level
Total cholesterol level
LDL cholesterol level
HDL cholesterol level

Dosage Regimens—All patients receive treatment medication once daily, in the morning one hour before breakfast. At each dosing, the prescribed number of tablets will be taken orally, with water, to allow for the following dosing levels:

| | |
|---|---|
| Study Drugs | Dosing Regimen |

PLACEBO LEAD-IN

| | |
|---|---|
| 1 Placebo Tablet = | Placebo for 250 mg P5P, OD |
| | (morning) |

ACTIVE

| | |
|---|---|
| 1 P5P 250 mg Tablet = | 250 mg P5P, OD (morning) |
| 2 P5P 250 mg Tablets = | 500 mg P5P, OD (morning) |
| 3 P5P 250 mg Tablets = | 750 mg P5P, OD (morning) |

PLACEBO LEAD-OUT

| | |
|---|---|
| Placebo Tablet = | Placebo for 250 mg P5P, OD |
| | (morning) |

The first dose of study medication is administered at clinic under the supervision of clinic personnel. Patients previously on anti-lipid therapy continued their anti-lipid regime unchanged throughout the study.

Duration of Treatment—Each patient is administered placebo or active treatment for a total period of 14 weeks after a successful eligibility assessment.

Results—As shown in Table 7, patients on concomitant P5P and statin treatment had improved lipid profiles at the end of the 14 week study. On average, concomitant P5P and a statin therapy reduced triglyceride levels and total cholesterol. In all but one patient, P5P and a statin therapy lowered LDL levels, increased HDL levels and improved the LDL:HDL ratio.

TABLE 7
Concomitant P5P and Statin Therapy

| | Triglyc- | | Total | | LDL | | HDL | | Chol:HDL | |
|---|---|---|---|---|---|---|---|---|---|---|
| | erides | | cholesterol | | cholesterol | | cholesterol | | ratio | |
| | V.1 | V.12 | V.1 | V.12 | V.1 | V.12 | V.1 | V.12 | V.1 | V.12 |
| Lipitor | | | | | | | | | | |
| 17 | 255 | 179 | 217 | 267 | 123 | 183 | 43 | 48 | 5.0 | 5.6 |
| 27 | 96 | 106 | 136 | 159 | 55 | 63 | 62 | 75 | 2.2 | 2.1 |
| Pravachol | | | | | | | | | | |
| 24 | 667 | 365 | 313 | 243 | | | 33 | 29 | 9.5 | 8.4 |
| Zocor | | | | | | | | | | |
| 23 | 164 | 212 | 152 | 145 | 80 | 68 | 39 | 35 | 3.9 | 4.1 |
| 25 | 222 | 187 | 272 | 230 | 185 | 153 | 43 | 40 | 6.3 | 5.8 |
| AVERAGE | 281 | 210 | 218 | 209 | 111 | 117 | 44 | 45 | 5.4 | 5.2 |
| (all | | | | | | | | | | |
| statins) | | | | | | | | | | |
| % change | | −25.3 | | −4.2 | | 5.4 | | 3.2 | | −3.3 |

V1: 1st visit, Week One, Day 1
V12: Last Visit, Week 14, Day 98

Reference Values:

Triglycerides: <150 mg/dL is normal

Total cholesterol: <200 mg/dL is normal

LDL cholesterol: <130 mg/dL is normal

HDL cholesterol: >or =40 mg/dL is normal

HDL cholesterol ratio: <4.4 is normal

On average, hypertensive patients taking P5P and a statin also had reductions in systolic and diastolic blood pressure at the end of the study.

### EXAMPLE FoUR

### P5P and Simvastatin Treatment Improves Lipid Profiles in Hypercholesteremic Rabbits

The study determines the potential anti-atherogenic effects of P5P alone and in combination with simvastatin in a rabbit hypercholesteremic model. The study compares the effects of PSP and simvastatin alone and in combination on atherosclerotic lesion formation, lipid profile (total cholesterol, LDL, HDL, triglycerides and oxidized LDL), homocysteine levels and various markers of inflammation (CRP, IL-1β,p, IL-6, IFN-γ and TNF-α).

Animals—Male New Zealand white rabbits 2.0-3.0 kg.

Animal Feed—The normocholesterol diet is Co-op Complete Rabbit Feed manufactured by Federated Co-operatives Limited (Saskatchewan). The 2% cholesterol diet is Test Diet 0009459 MRab/2% Chol 3/16 manufactured by Purina Mills (USA).

The rabbits are fed the normocholesterol and 2% cholesterol diet ad libitum. All animals receive a timothy cube after blood sampling.

Study Design—Prior to the start of the study, rabbits are randomly assigned to one of the following groups (n=9 per group):
Group 1: Normocholesterol diet (normal)
Group 2: Hypercholesterol diet (control)
Group 3: Hypercholesterol diet+P5P 10 mg/kg/day
Group 4: Hypercholesterol diet+simvastatin 5 mg/kg/day
Group 5: Hypercholesterol diet+P5P 10 mg/kg/day+simvastatin 5 mg/kg/day

The rabbits are fed the normocholesterol or hypercholesterol diet for a total of 8 weeks. The normocholesterol rabbits are treated once per day with 1 ml of ethanol and 5 ml of RO water via a nasogastric tube. The hypercholesterol animals are treated once per day with either P5P (10 mg/kg), simvastatin (5 mg/kg) or a combination of P5P and simvastatin (P5P 10 mg/kg; simvastatin 5 mg/kg) via a nasogastric tube. The P5P is dissolved in 5 ml of 0.05 N NaOH so that the pH of the solution is approximately 7. The simvastatin is dissolved in 1 ml of anhydrous ethanol.

TABLE 7
Test Compound Diluents

| Group | Diluent (small vial) | Diluent (large vial) |
|---|---|---|
| Normo | 1 ml ethanol | 5 ml RO water |
| Hyper | 1 ml ethanol | 5 ml RO water |
| Hyper + P5P | 1 ml ethanol | 5 ml 0.05 N NaOH |
| Hyper + Sim | 1 ml ethanol | 5 ml RO water |
| Hyper + Sim + P5P | 1 ml ethanol | 5 ml 0.05 N NaOH |

Five ml of blood is collected from the marginal ear vein of the rabbits at 0, 2, 4 and 6 weeks. Prior to blood collection the animals are sedated with 1 mg/kg of acepromazine. Following collection, the serum is separated. The serum is aliquoted (500 μl) and frozen at −80° C. until used for the assaying various lipids (total cholesterol, LDL, HDL, triglycerides, oxidized LDL) and cytokines (CRP, IL-1β, IL-6, IFN-γ and TNF-α).

After 8 weeks the animals are anesthetized and 10 ml of blood is collected. The animals are euthanized and the heart and thoracic aorta removed. The hearts and aortas are frozen in liquid nitrogen or an ethanol dry ice bath and stored at −80° C. until analyzed.

Time Lines

| | | | | ♥ |
|---|---|---|---|---|
| ♦ | ♦ | ♦ | ♦ | ♦ |
| 0 wk | 2 wk | 4 wk | 6 wk | 8 wk |
| ↑ | | | | ↑ |
| Start Treatment | | | | End Treatment |

♦ = blood collection
♥ = tissue collection

Test Articles —P5P (pyridoxal 5′-phosphate monohydrate) is purchased from Sigma (P82870). Simvastatin is obtained from ACIC Fine Chemicals Inc. Two test compound vials are provided for each animal for each day. The first vial (small) is diluted in 1 ml of anhydrous ethanol. The second vial is diluted in either 5 ml of RO water or 5 ml of 0.05 N NaOH. The diluent for each vial is clearly marked on the vial. The test solutions are prepared fresh each day just before they are administered to the animals. The stock P5P is stored in a refrigerator at 4° C. under low light conditions. The simvastatin is stored following the manufacture recommendations. The individual test vials are stored in a refrigerator at 4° C. under low light conditions.

Test Article(s) Dose, Route of Administration and Duration—The rabbits are treated orally with the drugs, once per day for a total of 8 weeks. The contents of vial one (small; 1 ml) are given to the animals first, followed by the contents of vial 2 (large; 5 ml). The test compound treatments are followed by a 5 ml RO water chaser.

Results—Treatment with P5P and the HMG Co reductase inhibitor results in lower LDL and triglyceride levels, higher HDL levels, lower homocysteine levels, and in general better cardiovascular health.

1. A pharmaceutical composition comprising: (a) a HMG CoA reductase inhibitor; (b) pyridoxal 5′-phosphate; and (c) a pharmaceutically acceptable carrier.

2. The pharmaceutical composition according to claim 1 wherein the HMG CoA reductase inhibitor is selected from a group consisting: pravatstatin, lovastatin, fluvastatin, atorvastatin, simvastatin, rosuvastatin, velostatin, fluindostatin, and a mixture thereof.

3-7. (canceled)

8. A method for treating a patient at risk of cardiovascular disease comprising administering a therapeutically effective dose of the pharmaceutical composition according to claim 1.

9. The method according to claim 8, wherein the patient is susceptible to hepatotoxicity.

10. The method according to claim 8 wherein the cardiovascular disease is selected from a group consisting: congestive heart failure, myocardial ischemia, arrhythmia, myocardial infarction, ischemic stroke, hemorrhagic stroke, coronary artery disease, hypertension, atherosclerosis, aneurysm, peripheral artery disease (PAD), thrombophlebitis, diseases of the heart lining, diseases of the heart muscle, carditis, congestive heart failure, endocarditis, ischemic heart disease, valvular heart disease, arteriosclerosis, acute coronary syndrome (ACS), deep vein thrombosis (DVT), Kawazaki disease, high cholesterol, restinosis, late vein graft failure and heart transplant.

11. The method according to claim 8 wherein the dose of the HMG CoA reductase inhibitor is between 0.1 and 1000 mg per day.

12. The method according to claim 8 wherein the dose of the HMG CoA reductase inhibitor is 10 mg per day.

13. The method according to claim 8 wherein the dose of the HMG CoA reductase inhibitor is 20 mg per day.

14. The method according to claim 8 wherein the dose of the pyridoxal 5′-phosphate is between 0.1 to 50 mg/kg per day.

15. The method according to claim 8 wherein the dose of pyridoxal 5′-phosphate is between 1 to 15 mg/kg per day.

16. A method of treating a patient at risk for diabetes comprising administering a therapeutically effective dose of the pharmaceutical composition according to claim 1.

17. A method for treating a patient at risk of Alzheimer's disease comprising administering a therapeutically effective dose of the pharmaceutical composition according to claim 1.

18. A method for treating a patient at risk of osteoporosis comprising administering a therapeutically effective dose of the pharmaceutical composition according to claim 1.

19. A method of treating hypercholesterolemia in a patient, comprising administering a therapeutically effective dose of pyridoxal-5′-phosphate.

20-23. (canceled)

24. A method for treating a patient at risk of cardiovascular disease, said patient being administered a HMG CoA reductase inhibitor, comprising administering a therapeutically effective dose of pyridoxal 5′-phosphate and a pharmaceutically acceptable carrier.

25. The method according to claim 24 wherein the HMG CoA reductase inhibitor is selected from a group consisting: pravastatin, lovastatin, fluvastatin, atorvastatin, simvastatin, rosuvastatin, velostatin, fluindostatin, and a mixture thereof.

26-30. (canceled)

31. The method according to claim 24 wherein the patient is susceptible to hepatotoxicity.

32. The method according to claim 24 wherein the cardiovascular disease is selected from a group consisting: congestive heart failure, myocardial ischemia, arrhythmia, myocardial infarction, ischemic stroke, hemorrhagic stroke, coronary artery disease, hypertension, atherosclerosis, aneurysm, peripheral artery disease (PAD), thrombophlebitis, diseases of the heart lining, diseases of the heart muscle, carditis, congestive heart failure, endocarditis, ischemic heart disease, valvular heart disease, arteriosclerosis, acute coronary syndrome (ACS), deep vein thrombosis (DVT), Kawazaki disease, high cholesterol, restinosis, late vein graft failure and heart transplant.

33. The method according to claim 24 wherein the dose of the HMG CoA reductase inhibitor is between 0.1 and 1000 mg per day.

34. The method according to claim 24 wherein the dose of the HMG CoA reductase inhibitor is 10 mg per day.

35. The method according to claim 24 wherein the dose of the HMG CoA reductase inhibitor is 20 mg per day.

36. The method according to claim 24 wherein the dose of pyridoxal 5′-phosphate is between 0.1 to 50 mg/kg per day.

37. The method according to claim 24 wherein the dose of pyridoxal 5′-phosphate is between 1 to 15 mg/kg per day.
